(19) United States
(12) Patent Application Publication
(10) Pub. No.: US 2026/0061405 A1
(43) Pub. Date: Mar. 5, 2026

(54) METHOD FOR DEHYDRATING A FEEDSTOCK COMPRISING AN ALCOHOL FOR THE PRODUCTION OF ALKENES

(71) Applicants: IFP ENERGIES NOUVELLES, Rueil-Malmaison (FR); TOTALENERGIES ONETECH, Courbevoie (FR)

(72) Inventors: Sylvie MAURY, Rueil-Malmaison Cedex (FR); Vincent COUPARD, Rueil-Malmaison Cedex (FR); Adrien BERLIET, Rueil-Malmaison Cedex (FR); Véronique DELATTRE, Rueil-Malmaison Cedex (FR); Delphine MINOUX-VENTELON, Seneffe (BE)

(73) Assignees: IFP ENERGIES NOUVELLES, Rueil-Malmaison (FR); TOTALENERGIES ONETECH, Courbevoie (FR)

(21) Appl. No.: 19/106,075

(22) PCT Filed: Aug. 24, 2023
(86) PCT No.: PCT/EP2023/073297

§ 371 Date: Feb. 24, 2025

(30) Foreign Application Priority Data
Aug. 25, 2022 (FR) FR2208531

(51) Int. Cl. B01J 29/65; B01J 35/61; B01J 35/63; C07C 1/24; C07C 11/02; C07C 31/12; C12P 7/04

(52) U.S. Cl. CPC B01J 29/65; B01J 35/613; B01J 35/633; C07C 1/24; C07C 11/02; C07C 31/12; C12P 7/04; C12P 2203/00

(57) ABSTRACT

The present invention relates to a method for the isomerization dehydration of a feedstock comprising at least one primary monoalcohol, of formula R—CH₂—OH, in which R is a nonlinear alkyl radical of general formula CₙH₂ₙ₊₁ where n is an integer between 3 and 20, the method comprising an isomerization dehydration step carried out in the gas phase, at a weighted mean temperature of between 200 and 300° C., at a pressure of between 0.1 and 1 MPa, at a weight hourly space velocity (PPH) of between 1 and 25 h⁻¹, in the presence of a catalyst comprising at least one zeolite, wherein the zeolite has at least one series of channels, the pore opening of which is defined by a ring of eight oxygen atoms (8MR) and has a mesopore volume of 0.10 ml/g or greater.

### TECHNICAL FIELD OF THE INVENTION

The present invention relates to an improved process for production of alkenes from a feedstock comprising at least one primary monoalcohol, of formula R—CH₂—OH, in which R is a nonlinear alkyl radical of general formula CₙH₂ₙ₊₁ where n is an integer of between 3 and 20 (such as isobutanol). This feedstock may be obtained by chemical processes or by fermentative processes. This process employs a dehydration reaction in the presence of a catalyst based on a zeolite having particular textural and morphological characteristics.

The alkenes obtained, for example butenes and in particular isobutene, 1-butene and 2-butenes, are of great interest in the field of the petrochemical industry and organic synthesis.

### PRIOR ART

Butenes are key molecules in petrochemistry, particularly for the synthesis of gasoline additives such as ETBE and MTBE. The great majority of the scientific publications concern the production of isobutene from linear butanols, which are more readily produced than isobutanol by conventional fermentative routes (ABE). Recent developments, however, have made it possible to greatly improve the fermentative yields of isobutanol, making this feedstock accessible and available at attractive cost.

The conversion of branched alcohols to alkenes, such as the conversion of isobutanol to butenes, is of great interest in the field of petrochemicals. The selectivity of the dehydration reaction in the presence of a solid catalyst and the stability of the catalyst in the presence of the water generated by the reaction remain parameters that the person skilled in the art constantly seeks to improve. Moreover, during alcohol dehydration, the alkenes generated can undergo oligomerization reactions, in particular at the acid sites of the dehydration catalysts, leading to deactivation of said catalysts (coke formation, pore clogging and poisoning of the acid sites). This well-known phenomenon in the presence of zeolitic catalysts and in the absence of hydrogen must be strictly limited in order to improve the lifetime of the catalysts and consequently the profitability of the alcohol dehydration process.

Moreover, the alcohols produced by fermentation of biomass or syngas contain impurities in the form of oxygen- and nitrogen-containing compounds produced during the processes of metabolization of the feedstocks into alcohols by yeasts. These compounds may have a deleterious effect on catalytic processes using biobased alcohols or products thereof, in particular by forming unwanted species responsible for deactivating dehydration or oligomerization catalysts, or by poisoning the active sites—in the event of these compounds being basic, they will be able to neutralize the acid sites of catalysts commonly used for dehydration, oligomerization or polymerization reactions, for example. Oxygen-containing compounds such as aldehydes, esters and ethers in particular can be partially eliminated by various pretreatments during the separation of the alcohol from the fermentation medium. An additional step can be carried out to remove the oxygen-containing compounds not separated during distillation. The basic nitrogen-containing compounds (amines, pyrazines), on account of their Brønsted or Lewis basic function, will poison the acid sites of the catalysts, causing deactivation thereof, and it is therefore preferable to remove them in order to increase the cycle time of the catalysts and to maintain the selectivity of the processes. Catalysts of moderate acidity risk greater deactivation in the presence of strong basic molecules.

Document WO2016046296 describes the modification of zeolites of structural type FER in powder form by treatment in the presence of organic acid or by ion exchange which makes it possible to lower the ratio of strong acid sites to weak acid sites to below 1. Catalysts are used in simultaneous dehydration and skeletal isomerization reactions of isobutanol. This treatment makes it possible to slightly limit the formation of coke on the catalysts, and above all to modify the nature thereof (C/H) while maintaining good activity and good selectivity for butenes, but without improving them.

Chadwick et al. (Chadwick et al., Chem. Commun., 2010, 46, 4088-4090) test various zeolites: Theta-1, ZSM-23, ferrierite (Si/Al=10 or 22.5) and ZSM-5 (10MR), at 400° C., for simultaneous dehydration and isomerization of n-butanol to give isobutene. They demonstrate a loss of isomerizing activity of ferrierite for isobutene formation over time and attribute it to a negative effect of the water formed by the dehydration reaction, leading to its dealumination. This is not observed with the other tested zeolites, ferrierite being the least stable zeolite among the zeolites tested.

Document WO18087031 describes the shaping of a ferrierite zeolite with an aluminous binder and its use in the low-temperature alcohol dehydration reaction. WO18087031 shows that performance is improved when the tested ferrierite of Si/Al ratio 20 is shaped with alumina as compared to being shaped with silica: conversions achieved with catalysts based on ferrierite and alumina at 250° C., WHSV 7 h⁻¹ are greater than 90% and the ratio of linear butenes to total butenes is about 87%, whereas with a catalyst based on ferrierite and a silica binder and a WHSV of 7 h⁻¹ it is 72.5% and the ratio of linear butenes to total butenes is about 82%. No mention is made of the stability of the catalysts.

Van Daele et al. (Applied Catalysis B: Environmental 284 (2021) 119699) sought to elucidate the mechanism of the isobutanol dehydration reaction over various zeolites and in particular over ferrierite zeolites differing in Si/Al. His objective was to understand the exceptional selectivity for linear butenes obtained from the branched C₄ alcohol isobutanol. For this, the ratios of (surface acidity/total acidity) and nature of acidity (Lewis acidity/Brønsted acidity) were modified by treatment of different ferrierites. Ferrierites of different morphologies to adjust the quantity of pores, the surface area and the external acidity of the crystals were synthesized. An effect of the external acidity of the zeolites on the activity was demonstrated, but no clear correlation could be established with the selectivity for n-butenes or the stability of the catalysts. A loss of activity of 15% was observed in 4 h with a ferrierite in the form of nano-needles with an external surface area of 240 m²/g, while it was 33% with nano-sheets of ferrierite with an external surface area of 42 m²/g. The studies were conducted under very low partial pressure of alcohol (45 mbar), at 250° C. and a WHSV of 100 h⁻¹; conversion levels are less than 60%.

The aim of the present invention is to overcome the drawbacks of the prior art of processes for dehydrating branched alcohols using zeolites to obtain alkenes, by providing a process improved in particular from the standpoint of:
the stability of the dehydration catalyst and its lifetime, by limiting its deactivation;
the profitability of the dehydration process in terms of catalyst activity, selectivity and yield of target products: linear alkenes.

### SUMMARY OF THE INVENTION

The present invention relates to a process for isomerizing dehydration of a feedstock comprising at least one primary monoalcohol, of formula R—CH₂—OH, in which R is a nonlinear alkyl radical of general formula CₙH₂ₙ₊₁ where n is an integer of between 3 and 20, said process comprising a gas-phase isomerizing dehydration step at a weighted average temperature of between 200 and 300° C., at a pressure of between 0.1 and 1 MPa, at a weight hourly space velocity (WHSV) of between 1 and 25 h⁻¹, in the presence of a catalyst comprising at least one zeolite, wherein said zeolite has at least one series of channels whose pore opening is defined by a ring of 8 oxygen atoms (8MR) and has a mesopore volume of 0.10 ml/g or more.

The advantage of the process according to the invention lies in the fact that the use of a catalyst comprising a zeolite according to the invention and possessing the particular textural properties makes it possible to obtain improved performances, especially in terms of stability but also of selectivity for linear alkenes and of conversion of the feedstock.

The catalyst is active at temperatures lower than those normally used and remains stable. The possibility of working at temperatures below 300° C. while maintaining a total conversion of the alcohol is also an advantage of the invention.

The linear alkenes obtained are of great interest in the field of the petrochemical industry and organic synthesis. The controlled oligomerization of these alkenes may also make it possible to produce aviation fuels and thus to obtain BioJet.

The applicant has demonstrated, surprisingly, that the use of a catalyst comprising a zeolite according to the invention and possessing particular textural properties in a process for isomerizing dehydration of a feedstock comprising a primary monoalcohol makes it possible to obtain a conversion rate of said alcohol which is much higher than that obtained with the zeolites used in the processes of the prior art, together with low deactivation of the zeolite catalyst.

The applicant has also demonstrated, surprisingly, that the use of a catalyst comprising a zeolite according to the invention, and in particular possessing particular textural properties (in particular a particular mesopore volume), in the isomerizing dehydration reaction, makes it possible to capture basic nitrogenous impurities present in the alcoholic feedstock and to produce an alkene effluent with a low nitrogen-containing compound content, in particular upstream of an oligomerization step, thus protecting from deactivation the catalyst downstream of the isomerizing dehydration, in particular the oligomerization catalyst. The advantage of the use of a catalyst according to the invention, which is very active, is that it allows simultaneous capture of basic nitrogen-containing molecules and isomerizing dehydration of the alcohol at the temperature of the dehydration reaction. This makes it possible to avoid an intermediate step of purifying the effluent comprising the alkenes obtained upstream of the oligomerization step.

### DESCRIPTION OF EMBODIMENTS

According to the present invention, the expressions “of between . . . and . . . ” and “between . . . and . . . ” are equivalent and mean that the limiting values of the interval are included in the range of values described. If this is not the case and the limiting values are not included in the range described, such a detail will be provided by the present invention.

In the sense of the present invention, the various parameter ranges for a given step, such as the pressure ranges and the temperature ranges, may be used alone or in combination. For example, in the sense of the present invention, a range of preferred pressure values may be combined with a more preferred range of temperature values.

In the following, particular embodiments of the invention may be described. They may be implemented separately or combined with each other, without limitation of combinations where technically feasible.

### Feedstock

According to the invention, the feedstock treated in the process according to the invention is a feedstock comprising, preferably consisting of, at least one primary monoalcohol, of formula R—CH₂—OH, in which R is a nonlinear alkyl radical of general formula CₙH₂ₙ₊₁ where n is an integer of between 3 and 20 (such as isobutanol), alone or as a mixture.

In the remainder of the specification, the term alkyl denotes a hydrocarbon compound of general formula CₙH₂ₙ₊₁ where n is an integer of between 3 and 20, preferably between 3 and 10, preferentially between 3 and 5, or even equal to 4.

In one embodiment, the feedstock comprises at least 40% by weight of primary monoalcohol relative to the total weight of said feedstock.

In one embodiment, the feedstock comprises at least 70% by weight of primary monoalcohol relative to the total weight of said feedstock.

In one embodiment, the feedstock comprises at least 90% by weight of primary monoalcohol relative to the total weight of said feedstock.

Mention may be made, as primary monoalcohol according to the invention, of isobutanol; 2-methylbutan-1-ol; 2,2-dimethylpropan-1-ol; 2-methylpentan-1-ol; 2,2-dimethylbutan-1-ol; 2-ethylbutan-1-ol. They may be alone or in a mixture.

Said primary monoalcohol is preferably isobutanol or 2-methyl-1-butanol, taken alone or as a mixture. Very preferably, said primary monoalcohol is isobutanol. Preferably, the feedstock comprises between 40% and 100% by weight, preferably between 70% and 100% by weight, preferentially between 90% and 100% by weight of isobutanol.

Said feedstock may originate from chemical or biochemical processes, for example fermentative processes. In particular, this feedstock may be derived from at least one, in particular lignocellulosic, biomass fermentation process.

Said feedstock may contain water, in particular up to 60% by weight of water, preferably up to 30% of water, preferentially up to 10% by weight of water. It may also include inorganic (such as Na, Ca, P, Al, Si, K, SO₄) and organic impurities (such as methanol, ethanol, n-butanol, aldehydes, ketones, and the corresponding acids, for example furoic, acetic, isobutyric acid).

Said feedstock may contain nitrogenous impurities, in particular between 5 and 100 ppm of total nitrogen.

### Process

According to the invention, the process comprises a step of isomerizing dehydration of the feedstock comprising at least one primary monoalcohol of formula R—CH₂—OH, preferably carried out in the gas phase, at a weighted average temperature of between 200 and 300° C., preferably between 210 and 280° C., very preferably between 230 and 270° C., at a pressure of between 0.1 and 1.0 MPa, preferably between 0.3 and 1.0 MPa, very preferably between 0.5 and 1.0 MPa, at a weight hourly space velocity (WHSV) of between 1 and 25 h⁻¹, preferably between 1 and 20 h⁻¹, very preferably between 1 and 18 h⁻¹, in the presence of a dehydration catalyst.

Advantageously, the dehydration is carried out in a reactor or series of reactors comprising at least one catalytic bed.

WHSV is understood to correspond to the weight hourly space velocity. By weight hourly space velocity (WHSV) is meant the mass flow rate of primary monoalcohol of the feedstock (considered in dry condition) at the reactor inlet, divided by the mass of catalyst in said reactor.

Weighted average temperature (denoted WAT) means the average of the temperature in the catalytic bed, the bed being the set of beds present in the reactor, the beds in which the catalytic reaction takes place, as calculated along the axis of the flow in said bed. For a bed of length L and surface area S, the reactive mixture flowing along the longitudinal axis x of that bed, the inlet to the catalytic bed forming the origin of the axis (x=0), the weighted average temperature, denoted WAT, is expressed according to the following formula:

$$WAT = \frac{1}{L} {\int }_{0}^{L} T ( x ) dx$$

Since the reaction is endothermic and the reactor operates either in isothermal mode or in adiabatic mode, the weighted average temperature will be representative of the reaction temperature.

The reaction advantageously takes place in one or more reactors, for example isothermal or adiabatic reactors, which are arranged in particular in series or parallel, preferably in series, and each reactor is operated under particular or identical conditions. A person skilled in the art will be able to adjust the choice of operating conditions (pressure, temperature WAT, residence time) of each reactor as a function of the feedstock in order to obtain optimal conversion and the desired selectivity for linear olefins.

Preferably, the dehydration catalyst is disposed in one or more fixed beds, which may be operated in upflow, downflow or radial flow mode.

Since the dehydration reaction is endothermic, the dehydration step advantageously comprises an input of heat, the heat input being carried out by any heating means known to those skilled in the art.

Preferably, before being brought into contact with the feedstock to be treated, the dehydration catalyst is activated by any means known to those skilled in the art, for example by heat treatment in air.

The process according to the invention makes it possible to work at low temperature, in particular at a temperature of less than or equal to 300° C., preferably less than or equal to 270° C. The advantage of working at low temperature in the process according to the invention makes it possible to avoid local overheating of the alcohol (excessive temperature in contact with the metal surface of the reactor or the feedstock transport lines), with the potential risk of degradation of the primary monoalcohol, such as isobutanol; and to reduce consumption of utilities and cost of operation. The process according to this embodiment is therefore very economically advantageous.

### Dehydration Catalyst

According to the invention, the dehydration catalyst used comprises at least one zeolite which has at least one series of channels whose pore opening is defined by a ring of 8 oxygen atoms (8MR) and which has a mesopore volume of greater than or equal to 0.10 ml/g.

According to one embodiment, said zeolite may also advantageously have at least one series of channels whose pore opening is defined by a ring containing 10 oxygen atoms (10MR). These series of channels are defined in the classification “Atlas of Zeolite Framework Types”, Ch. Baerlocher, L. B. McCusker, D. H. Olson, 6th edition, Elsevier, 2007, Elsevier.

Said zeolite is advantageously chosen from zeolites having 8 and 10MR channels such as the zeolites of structural type FER and MFS, taken alone or as a mixture. The zeolite is more advantageously chosen, in the FER type, from ferrierite, FU-9, ISI-6, NU-23 and ZSM-35 zeolites, and for the MFS type it is the ZSM-57 zeolite, taken alone or as a mixture. Said zeolite is very advantageously of FER type and preferably is ferrierite. Preferably, said zeolite consists of ferrierite.

In one embodiment, the zeolite has a mesopore volume of greater than or equal to 0.15 ml/g.

In one embodiment, the zeolite has a mesopore volume of greater than or equal to 0.18 ml/g.

In one embodiment, the zeolite has a mesopore volume of greater than or equal to 0.20 ml/g, preferably greater than or equal to 0.22 ml/g, preferentially greater than or equal to 0.24 ml/g.

Preferably, the zeolite has a mesopore volume of less than or equal to 0.50 ml/g, preferably less than or equal to 0.40 ml/g, preferentially less than or equal to 0.35 ml/g, with preference less than or equal to 0.30 ml/g.

In one embodiment, the zeolite has a mesopore volume of between 0.18 and 0.50 ml/g.

In one embodiment, the zeolite has a mesopore volume of between 0.20 and 0.40 ml/g.

In one embodiment, the zeolite has a mesopore volume of between 0.22 and 0.35 ml/g.

In one embodiment, the zeolite has a mesopore volume of between 0.24 and 0.30 ml/g.

In one embodiment, the zeolite has an Si/Al molar ratio of between 5 and 45.

In one embodiment, the zeolite has an Si/Al molar ratio of between 5 and 30.

In one embodiment, the zeolite has an Si/Al molar ratio of between 8 and 20.

In one embodiment, the zeolite has an Si/Al molar ratio of between 9 and 15.

In one embodiment, the zeolite has an Si/Al molar ratio of between 11 and 13.

In one embodiment, the zeolite has a micropore volume of between 0.100 and 0.150 ml/g.

In one embodiment, the zeolite has a micropore volume of between 0.110 and 0.145 ml/g.

In one embodiment, the zeolite has a micropore volume of between 0.120 and 0.140 ml/g.

In one embodiment, the zeolite has a micropore volume of between 0.130 and 0.140 ml/g.

In one embodiment, the zeolite has a micropore volume of between 0.133 and 0.138 ml/g.

In one embodiment, the zeolite has an external surface area of between 10 and 70 m²/g.

In one embodiment, the zeolite has an external surface area of between 20 and 65 m²/g.

In one embodiment, the zeolite has an external surface area of between 30 and 60 m²/g.

In one embodiment, the zeolite has an external surface area of between 35 and 55 m²/g.

In one embodiment, the zeolite has an external surface area of between 45 and 50 m²/g.

In one embodiment, the zeolite has an average crystal size of less than or equal to 100 nm.

In one embodiment, the zeolite has an average crystal size of between 10 and 100 nm.

In one embodiment, the zeolite has an average crystal size of between 30 and 95 nm.

In one embodiment, the zeolite has an average crystal size of between 40 and 90 nm.

In one embodiment, the zeolite has an average crystal size of between 50 and 85 nm.

In one embodiment, the zeolite has an average crystal size of between 60 and 80 nm.

In one embodiment, the zeolite has a BET surface area of greater than or equal to 400 m²/g.

In one embodiment, the zeolite has a BET surface area of between 405 and 450 m²/g.

In one embodiment, the zeolite has a BET surface area of between 410 and 440 m²/g.

In one embodiment, the zeolite has a BET surface area of between 415 and 430 m²/g.

The crystals of the zeolites according to the invention have a rounded and elongated shape, in particular an oblong or spheroidal shape.

Advantageously, the zeolite crystals according to the invention are not in the form of needles or platelets.

The crystals of the zeolites according to the invention are arranged in the form of aggregates, preferably in spherical form, unlike the zeolites used in the processes of the prior art.

The catalyst may be in powder form or shaped.

Advantageously, the dehydration catalyst has a zeolite content of at least 50% by weight, preferably of between 55% and 90% by weight, very preferably between 60% and 80% by weight relative to the total weight of said dehydration catalyst.

In one embodiment, the dehydration catalyst comprises no metals.

“No metals” means that there are no metals added during preparation.

According to one particular embodiment of the invention, the catalyst is shaped with a binder, which advantageously is inert for the intended reaction (isomerizing dehydration of a primary monoalcohol). The shaping of the catalyst with a binder makes it possible to obtain a catalyst of macroscopic size for which the person skilled in the art can adapt the physical properties (geometry, pore mass volume, etc.). Indeed, when the zeolite cannot be used industrially in powder form, the binder allows the final solid to be given a mechanical strength necessary for industrial use and an increased strength in the presence of water. The binder also allows the catalyst thus constituted to be used in a fixed bed in a reactor without giving too great a pressure loss.

The binder is preferably chosen from a silicic binder such as silica, an aluminous binder such as gamma-alumina, an AlPO₄, a clay, a zirconia, a Ti oxide, SiC, or mixtures thereof.

Preferably, the binder is a silicic or aluminous binder. Preferably, the binder is a silicic binder, consisting essentially of silica, meaning that the silicic binder consists of silica except for impurities, these impurities having no catalytic effect. In particular, said silica is an amorphous silica.

Preferably, the dehydration catalyst has a binder content of between 10% and 45% by weight, preferably between 20% and 40% by weight relative to the total weight of said catalyst.

The dehydration catalyst may be shaped in the form of extrudates, according to a geometry which is, for example, cylindrical or multilobal, in particular trilobal, or a quadrilobe.

The process according to the invention, which uses a dehydration catalyst comprising a zeolite having such morphological and textural characteristics, makes it possible to obtain optimized performances in isomerizing dehydration of a primary alcohol, in particular in terms of conversion of the alcohol and selectivity toward linear alkenes. In addition, such a dehydration catalyst also exhibits improved deactivation stability and therefore an increased lifetime, which makes it possible to achieve advantageous profitability of the isomerizing dehydration process.

### Description of Analytical Methods

To determine the micropore volume, the t method (of Lippens and De Boer) described in the periodical Journal of catalysis, (Studies on pore systems in catalysts V. The t method, J. Catal., 1965, 4(3), p. 319) is used. It is based on the comparison between the experimental isotherm of the microporous solid and the reference isotherm (nonporous solid) of the same chemical nature. From the Lippens-De Boer equation, the thickness t of the multilayer can be calculated with the following equation (called t-plot):

$$\text{?} = {( \frac{0.1399}{0.034 - log ( P / {P}_{\text{?}} )} )}^{\text{?}}$$

$$\text{?} \text{indicates text missing or illegible when filed}$$

where P/P0 is the relative pressure of nitrogen.

The micropore volume is calculated with the following equation:

$$V μ ( ml / g ) = D * Y$$

where Y is the y-axis at the origin of the t-plot curve and D is the density conversion factor (D=15.468×10⁻⁴, a coefficient to ensure the conversion of gas volume to liquid volume). The range of t chosen corresponds to a plateau on the nitrogen adsorbed volume versus thickness t curve and is between 0.4 and 0.8 nm.

The mesopore surface area, also called external surface area, here is calculated using the t-plot curve with the following equation:

$$Smesolext ( {m}^{2} / g ) = D * S$$

where S is the slope of the t-plot straight line and D is the density conversion factor (D=15.468×10⁻⁴, a coefficient to transform gas volume into liquid volume).

The mesopore volume is considered here to be equal to the total volume of nitrogen adsorbed at P/P0 max minus the mass micropore volume.

The specific surface area or BET surface area is determined by the BET method (Brunauer, Emmett and Teller) described in the periodical “The Journal of American Chemical Society”, 1938, 60, 309. It is based on the specificity of physical adsorption: multimolecular adsorption on sites of the same energy. On the basis of all the assumptions (equivalent sites, no lateral interaction between adsorbed molecules, each adsorbed molecule can serve as adsorption sites) set out in this theory, the specific surface area may be deduced from the volume of nitrogen adsorbed on the monolayer, Vm, by virtue of the following equation:

$$SBET ( {m}^{2} / g ) = 4.37 * Vm .$$

The average size of the zeolite crystals is measured by bright-field transmission electron microscopy or TEM. Histograms of crystal size were made from photographs taken by transmission microscopy in bright-field mode. The average size was determined from the measurement of 200 crystals. The crystals observed for the zeolite according to the invention are preferably oblong or spheroidal in shape. The representation of the crystal in the photo is its projection along the beam axis. The dimension employed is an estimate of the size of a crystal along the axis traversed by the beam shown in FIG. 1, an average then being calculated on a sample of 200 crystals.

The examples and figures which follow illustrate the invention, more particularly specific embodiments of the invention, without limiting its scope.

### LIST OF FIGURES

FIG. 1 schematically represents a crystal and the axis along which the electron microscopy beam travels through said crystal to calculate its size.

FIG. 2 represents a scanning electron microscope (SEM) view of the solid B according to the invention.

FIG. 3 represents a scanning electron microscope (SEM) view of the comparative solid C.

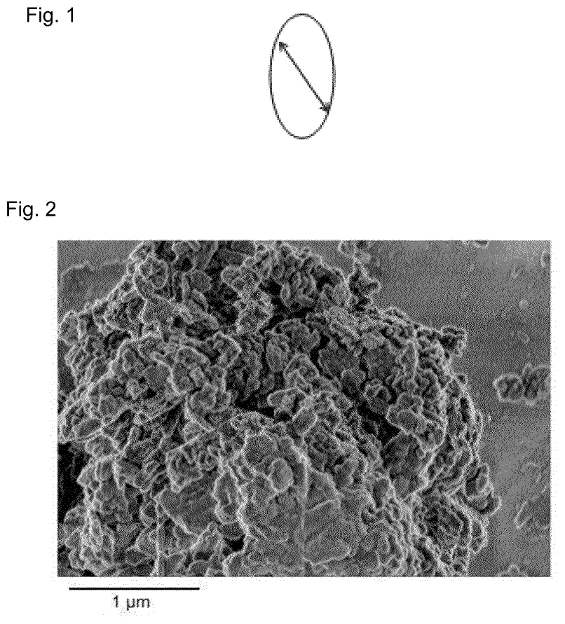

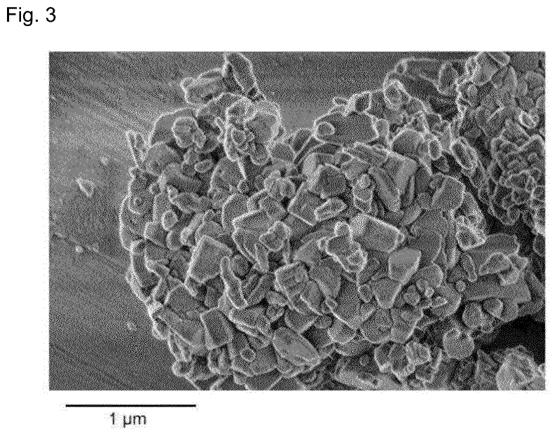

### EXAMPLES

### Example 1: Testing of Different Catalyst Samples in a Process of Dehydration of Isobutanol to n-Butenes

The dehydration step is carried out on a catalytic test EHD multireactor unit comprising fixed-bed reactors operating in downflow mode. The dehydration catalysts tested each comprise 100% by weight of a zeolite. Several zeolites were tested (cf. table 2).

The catalysts are charged separately into the reactors in powder form, previously pelletized, ground and then sieved to retain the 300-500 μm fraction. The catalysts are loaded into quartz reactors with an internal diameter of 4 mm, between two SiC beds. The catalysts are then activated at 450° C. under air purging for 6 h after a ramped temperature rise of 5° C./min. The temperature is then lowered to the test temperature under nitrogen in order to remove the air present in the system before injection of the isobutanol feedstock. The test is carried out at atmospheric pressure (i.e., about 0.1 MPa).

Different WHSVs are evaluated: 9, 6 and 3 h⁻¹ at two temperatures 240 and 250° C., a return point is carried out at the end of the test at 240° C. WHSV 9 h⁻¹. Each condition is maintained for 9 h, which makes it possible to acquire 5 chromatograms and to evaluate the deactivation of the zeolites. The test lasted 72 h. The sequence of conditions is presented below; these were applied to all the catalysts in a strictly identical way.

TABLE 1

| Condition No. | WHSV isobutanol (h⁻¹) | Temperature (° C.) |
|---|---|---|
| 1 | 9 | 240 |
| 2 | 6 | 240 |
| 3 | 3 | 240 |
| 4 | 9 | 250 |
| 5 | 6 | 250 |
| 6 | 3 | 250 |
| 7 | 9 | 240 |

The feedstock is an isobutanol/water mixture in a mass ratio of 99/1. It is vaporized in the SiC bed at the top of the reactor before coming into contact with the catalytic bed.

The analysis of the total effluent is carried out at the reactor outlet on an in-line gas chromatograph equipped with two capillary columns, which makes it possible to determine the conversion of isobutanol, the selectivities for different products and in particular the selectivity for butenes, and the fraction of linear butenes in the butene cut, an objective being to maximize this fraction.

The zeolites tested are presented in the following table:

TABLE 2

| Solid | Type of zeolite |
|---|---|
| A (according to the invention) | Ferrierite |
| B (according to the invention) | Ferrierite |
| C (comparative) | Ferrierite CP914C |
| D (comparative) | Ferrierite CP914 |
| E (comparative) | ZSM-5 CBV5020 |
| F (comparative) | ZSM-5 CBV2314 |

The textural properties of the zeolites evaluated are presented in the following table:

TABLE 3

| | Average size | | BET | | Micropore | Mesopore | | External |
|---|---|---|---|---|---|---|---|---|
| | of crystals | Observed | surface | Total | volume (Vμ) | volume = | Molar | surface |
| | as measured | crystal | area | volume | by t-plot | Vtotal − | Si/Al | area |
| Solid | by TEM (nm) | morphology | (m²/g) | (ml/g) | (ml/g) | Vμ (ml/g) | (FX) | (m²/g) |
| A | 100 | Spheroidal | 400 | 0.297 | 0.132 | 0.165 | 10.0 | 29.0 |
| B | 72 | Spheroidal | 425 | 0.401 | 0.136 | 0.265 | 12.5 | 47.0 |
| C | 170 | Faceted | 337 | 0.206 | 0.130 | 0.076 | 10.0 | 8.6 |
| | | polyhedral | | | | | | |
| D | 3000 | Platelets | 398 | 0.222 | 0.146 | 0.076 | 25.0 | 15.0 |
| E | 40 | Pebbles = | 408 | 0.399 | 0.115 | 0.284 | 25.0 | 42.0 |
| | | aggregates | | | | | | |
| | | of needles | | | | | | |
| | | and small | | | | | | |
| | | crystals | | | | | | |
| F | 500 | Faceted | 431 | 0.225 | 0.135 | 0.090 | 12.0 | 31.0 |
| | | polyhedral | | | | | | |

The catalytic results obtained at reaction times between 8-9 h of testing and after 70-72 h of testing, for the various zeolites tested, are presented in table 4 below. The catalytic results presented are the conversion of isobutanol, the proportion of N-butenes in the total butenes (N-butenes/total butenes) at the start of the test and after at least 70 h under load, the loss of activity (or deactivation) as a percentage per unit of time between the conversion at the initial point (at about 8-9 hours) and the conversion at the end point (i.e., around 70-72 hours) at iso conditions (240° C., WHSV 9 h⁻¹).

TABLE 4

| | | Isobutanol | N-butenes/ | Deactivation or loss of |
|---|---|---|---|---|
| | Time under | conversion | total | conversion |
| Solid | load (h) | (%) | butenes | (%/h) |
| A | 9.3 | 99.5 | 67.9 | 1.189 |
| | 72.4 | 24.4 | 58.9 | |
| B | 8.8 | 100.0 | 69.5 | 0.000 |
| | 72.0 | 100.0 | 68.2 | |
| C | 8.1 | 22.7 | 56.2 | 0.243 |
| | 71.3 | 7.4 | 46.9 | |
| D | 7.9 | 15.0 | 57.6 | 0.157 |
| | 71.0 | 5.0 | 45.0 | |
| E | 7.6 | 90.6 | 32.3 | 0.800 |
| | 70.8 | 40.0 | 34.9 | |
| F | 8.3 | 99.3 | 38.3 | 1.004 |
| | 71.5 | 35.9 | 39.0 | |

It is noted that of the four ferrierites tested (A, B, C and D), those according to the invention, which have a mesopore volume of greater than 0.10 ml/g (i.e., zeolites A and B), exhibit a much higher activity after 8 to 10 h under load than those having a mesopore volume of less than 0.10 ml/g (C and D). For example, at a similar molar ratio (Si/Al of 10), ferrierite A has a higher initial activity than ferrierite C (99.5% conversion at about 9 h for zeolite A versus 22.7% conversion at about 8 h for zeolite C).

In terms of stability, ferrierite B according to the invention, having a mesopore volume of 0.265 ml/g (therefore greater than 200 ml/g, or even greater than 0.22 ml/g), and advantageously having an external surface area of between 35 and 55 m²/g, or even of between 45 and 50 m²/g, and an Si/Al molar ratio of 12.5, converts isobutanol optimally, since it converts isobutanol 100% for 72 h regardless of temperature and WHSV. This zeolite combines high initial and final conversions, a selectivity for linear butenes which is improved and quasi-stable over the test time compared with the other zeolites tested, and low deactivation (or even zero deactivation over 72 hours). Ferrierites C and D are little deactivated, but the initial conversion of isobutanol over these solids is less than 50% at 240° C.

### Example 2: Test of Capacity of the Catalysts to Capture Nitrogen-Containing Compounds

In this example, the feedstock is an isobutanol/water mixture in a mass ratio of 95/5 comprising 6 ppm of acetonitrile. The same apparatus is used. The feedstock is vaporized in the SiC bed at the top of the reactor before coming into contact with the catalytic bed. Various catalysts were tested: zeolites A, B, C, D, E and F described in example 1 and a gamma-alumina (cf. table 6). The gamma-alumina has a low total NH₃ acidity (300 μmol/g). The catalysts are prepared as described in example 1. The catalysts were tested under conditions strictly identical (except for the presence of nitrogen-containing compound) to those of example 1.

The following table shows the nitrogen, carbon and hydrogen contents of the different catalysts, analyzed after 72 h of reaction for the different catalysts tested:

TABLE 5

| Solid | N content (%) | H content (%) | C content (%) |
|---|---|---|---|
| A | 0.115 | 1.253 | 8.137 |
| B | 0.212 | 1.383 | 8.956 |
| C | 0.057 | 1.112 | 7.642 |
| D | 0.084 | 0.87 | 5.884 |
| E | 0.147 | 1.113 | 9.148 |
| F | 0.197 | 1.054 | 9.691 |
| gamma-Alumina | <0.004 | 0.9 | 3.600 |

It is noted that zeolites A to F have a significantly higher nitrogen content than that measured for the gamma-alumina, from which it may be deduced that the zeolites capture at least part of the nitrogenous impurities of the isobutanol feedstock.

Moreover, it appears that ferrierites A and B have a nitrogen content (respectively 0.115% and 0.212%) greater than that of ferrierites C and D, the nitrogen content of which is less than 0.10%. This indicates that the catalysts according to the invention, ferrierite B very significantly, possess a capacity to capture nitrogen-containing compounds that is superior to that of the ferrierite zeolites used in the processes of the prior art.

1. A process for isomerizing dehydration of a feedstock comprising at least one primary monoalcohol, of formula R—CH2—OH, in which R is a nonlinear alkyl radical of general formula CnH2n+1 where n is an integer of between 3 and 20, said process comprising a gas-phase isomerizing dehydration step at a weighted average temperature of between 200 and 300° C., at a pressure of between 0.1 and 1 MPa, at a weight hourly space velocity (WHSV) of between 1 and 25 h−1, in the presence of a catalyst comprising at least one zeolite, wherein said zeolite has at least one series of channels whose pore opening is defined by a ring of 8 oxygen atoms (8MR) and has a mesopore volume of greater than or equal to 0.10 ml/g.

2. The process as claimed in claim 1, wherein the zeolite has an Si/Al molar ratio of between 5 and 45.

3. The process as claimed in claim 1, wherein the zeolite has an external surface area of between 10 and 70 m²/g, preferably between 20 and 65 m²/g, very preferably between 35 and 55 m²/g.

4. The process as claimed in claim 1, wherein the zeolite has a micropore volume of between 0.100 and 0.150 ml/g.

5. The process as claimed in claim 1, wherein the zeolite also has a series of channels whose opening is of 10 oxygen atoms (10MR).

6. The process as claimed in claim 5, wherein the zeolite is of structural type FER or MFS.

7. The process as claimed in claim 6, wherein the zeolite is a ferrierite.

8. The process as claimed in claim 1, wherein the feedstock comprises from 90% to 100% by weight of primary monoalcohol relative to the total weight of said feedstock.

9. The process as claimed in claim 1, wherein the primary monoalcohol is isobutanol.

10. The process as claimed in claim 1, wherein the feedstock is derived from at least one lignocellulosic biomass fermentation process.
